(19) United States
(12) Patent Application Publication
(10) Pub. No.: US 2021/0154371 A1
(43) Pub. Date: May 27, 2021

(54) BIOCOMPATIBLE TEXTILE MESH AND TISSUE CONSTRUCTS FROM MANICARIA SACCIFERA, METHODS OF GROWING CELLS AND TISSUES, AND METHODS OF TREATING SUBJECTS WITH THE BIOCOMPATIBLE TEXTILE MESH AND TISSUE CONSTRUCTS

(71) Applicant: University of Florida Research Foundation, Inc., Gainesville, FL (US)

(72) Inventors: Josephine ALLEN, Gainesville, FL (US); Juan Claudio NINO, Gainesville, FL (US)

(21) Appl. No.: 16/953,849

(22) Filed: Nov. 20, 2020

Related U.S. Application Data
(60) Provisional application No. 62/939,485, filed on Nov. 22, 2019.

(51) Int. Cl. A61L 27/56; A61L 27/36; A61L 27/38; A61L 27/54

(52) U.S. Cl. CPC A61L 27/56; A61L 27/3641; A61L 27/54; A61L 27/3839; A61L 27/3886; A61L 27/3683

(57) ABSTRACT

Various embodiments of biocompatible textile mesh and tissue constructs from Manicaria saccifera, methods of growing cells and tissues using the Manicaria saccifera-based textile mesh/tissue scaffolds, and methods of treating subjects with the biocompatible textile mesh and tissue constructs are described. The mesh, constructs and methods can include a biocompatible textile mesh made from a naturally woven fiber mat from a Manicaria saccifera palm bract that has been treated to remove oils and lignin from the surface of palm fibers in the mat and seeded with a population of cells. An engineered, biocompatible tissue construct, a method of growing mammalian tissue in vivo, and a method of treating a subject are also described.

### CROSS-REFERENCE TO RELATED APPLICATION

This application claims the benefit of and priority to U.S. Provisional Application No. 62/939,485, titled “BIOCOMPATIBLE TEXTILE MESH AND TISSUE CONSTRUCTS FROM MANICARIA SACCIFERA, METHODS OF GROWING CELLS AND TISSUES, AND METHODS OF TREATING SUBJECTS WITH THE BIOCOMPATIBLE TEXTILE MESH AND TISSUE CONSTRUCTS,” filed on Nov. 22, 2019, the contents of which are hereby incorporated herein by reference in its entirely.

### BACKGROUND

In recent years, natural materials have gained in notoriety for their use in engineering applications as sustainable alternatives. This interest has not eluded tissue engineering, which requires scaffolds with complex architectures, low-cost, and scalability to transition from the benchtop to the clinic. Plant-based materials offer such properties. Like mammalian tissue, plant tissue is composed of hierarchical structures. Of note is the similarity in mammalian and plant vascular architecture. While some decellularized plants have been investigated as scaffolds to demonstrate perfusion of plant vasculature by mammalian endothelial cells and cardiomyocytes, such plant tissues require steps such as decellularization and/or functionalization prior to use. Furthermore, bioactivity and immunogenicity of plant materials with mammalian/human cells and tissues varies widely among different plant species, thus making immunogenicity and biocompatibility of plant scaffolding materials unpredictable.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1A-1D are images illustrating examples of Manicaria saccifera fiber mats before (FIGS. 1A, 1B) and after (FIGS. 1C, 1D) alkali treating according to various embodiments of the present disclosure showing that following treatment, the fiber surface appears smoother and the color is faded.

FIGS. 2A-2C illustrate stress-strain curves for fiber tensile testing of examples of individual Manicaria saccifera fibers: untreated fibers (FIG. 2A), alkali treated (FIG. 2B), and alkali treated and autoclaved (FIG. 2C) according to various embodiments of the present disclosure. Fibers were pulled at a strain rate of 10 mm/min (0.17 mm/s). The degree of variability observed in the stress-strain curves is common in natural fibers.

FIG. 3 illustrates transverse tensile loading of Manicaria saccifera fiber mats according to various embodiments of the present disclosure. The fiber mats displayed upwards of 100% strain.

FIG. 4 is a panel of images illustrating extended culture of different cell types (NIH3T3 cells, HAoSMC cells, and HADSC cells) over time on Manicaria saccifera fiber mats according to various embodiments of the present disclosure. All three cell types were able to attach to the fibers after 1 day. Over a period of 3 weeks, the cells engulfed the fibers and formed webbings between proximal fibers examples of which are indicated by white arrows.

FIG. 5 provides phase contrast (Ph) and fluorescence (Fl) images illustrating cell viability on Manicaria saccifera fiber mats according to various embodiments of the present disclosure. At 21 days, each cell type showed robust cellular viability at multiple levels of thickness indicated by the strong green fluorescence (lower panels, Fl) from the calcein AM live cell staining. There were little to no dying or dead cells indicated by the absence of red ethidium homodimer-1 staining in the fluorescence (Fl) images. Phase contrast (Ph) images (upper panel) are shown to indicate the location of the fibers

FIGS. 6A-6D are images illustrating histological analysis of NIH/3T3 mouse fibroblasts cultured on Manicaria saccifera fiber mats according to various embodiments of the present disclosure. Sections showed cells aligned along the fiber axis and this alignment extended over several layers of cells (FIG. 6A). Additionally, some regions showed masses of randomly aligned cells surrounded by a layer of several cells aligned with the fiber axis (FIGS. 6A and 6B). Cellular bridging across fibers was also observed (FIGS. 6C and 6D). M=Manicaria saccifera fiber, *=NIH/3T3 mouse fibroblasts

FIGS. 7A-7D are a series of SEM micrographs of NIH3T3s (FIGS. 7A, 7B) and HADSC (FIGS. 7C, 7D) on Manicaria saccifera fibers according to various embodiments of the present disclosure. Bridging across fibers was observed by SEM (FIG. 7B). Fiber dimples with and without silicate particles were observed as marked by arrows (FIGS. 7B and 7D)

FIGS. 8A-8C are graphs illustrating in vitro immunological assessment by analysis of gene expression and protein expression by cells grown in various culture conditions and/or Manicaria saccifera fiber mats according to various embodiments of the present disclosure. Gene expression after 4 hrs (FIG. 8A) and 24 hrs (FIG. 8B) and protein expression after 24 hrs (FIG. 8C) of interleukin-1β (IL-1β), interleukin-8, (IL-8), and tumor necrosis factor α (TNFα). Statistical significance between treatments is denoted by *. Treatments were measured in triplicate.

FIG. 9 illustrates an Ashby plot comparing various natural (▪) and common synthetic (▴) materials for tissue engineering and of the Manicaria saccifera fibers/mats (●) according to various embodiments of the present disclosure. Each material is plotted as a function of the tensile strength and Young's modulus (stiffness). The mechanical properties of Manicaria saccifera individual fibers and fiber mats (●) are highlighted. The transverse fiber mat mechanical properties are shown using a segmented x and y axis in the figure. UT=untreated, AT=alkali treated, ATA=alkali treated and autoclaved, PU=polyurethane, PTFE=polytetrafluoroethylene, PMMA=polymethylmethacrylate, PE=polyethylene. (Some data compiled from references 9, and 27-30).

### DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

Any publications and patents cited in this specification that are incorporated by reference (as noted) are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of biochemistry, organic chemistry, molecular biology, biology, materials science, and the like, which are within the skill of the art. Such techniques are explained fully in the literature.

It must be noted that, as used in the specification and the appended embodiments, the singular forms “a,” “an,” and “the” include plural referents unless the context clearly dictates otherwise. Thus, for example, reference to “a cell” includes a plurality of cells. In this specification and in the embodiments that follow, reference will be made to a number of terms that shall be defined to have the following meanings unless a contrary intention is apparent.

As used herein, the following terms have the meanings ascribed to them unless specified otherwise. In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

Definitions

In describing the disclosed subject matter, the following terminology will be used in accordance with the definitions set forth below.

The term “biomaterial” as used in the present disclosure refers to living and/or naturally derived biological materials or cells (e.g., tissues, cells, or other biomolecules derived from a living organism) as well as biocompatible synthetic materials (e.g., man-made and/or engineered materials that can be used in conjunction with and without harm to living biomaterials (e.g., synthetic scaffolds for use with living cells).

As used herein, “isolated” means removed or separated from the native environment. An isolated compound or material (e.g., a cell, tissue, protein, etc.) indicates the compound or material is separated from its natural environment. Isolated compounds or materials are not necessarily purified.

The term “tissue” generally refers to a grouping of cells organized to cooperatively carry out a biological function and/or serve a biological purpose, such as forming all or part of an organ in an organism (e.g., connective tissue, endothelial tissue, muscle tissue, etc.). While a “tissue” generally includes a grouping of similar cells, or cells of all the same type, a tissue may also include cells of more than one type where the group of cells as a whole serve a common purpose.

“Cells” as used herein, includes both prokaryotic and eukaryotic cells, and both plant cells (including monocots and dicots) and animal cells (e.g., avian, amphibian, reptile, and mammalian cells). In some embodiments, mammalian cells (including but not limited to human, monkey, ape, dog, cat, mouse, rat, horse, goat, sheep, etc.) are preferred. Cells may reside in vitro or in vivo in a tissue in organ.

As used herein, the term “biocompatible” refers to the ability to co-exist with a living biological substance and/or biological system (e.g., a cell, cellular components, living tissue, organ, etc.) without exerting undue stress, toxicity, or adverse effects (e.g., significant immunogenic response, cell death, etc.) on the biological substance or system.

As used herein, the term “naturally woven” indicates that the fibers in the mat have a natural configuration where some of the fibers are interconnected and/or overlapping, and/or woven/twisted together, but does not indicate that the fibers have been woven by hand.

### Discussion

Briefly described, embodiments of the present disclosure encompass methods of growing cells and tissues using a biocompatible textile mesh of the present disclosure made from a naturally woven fiber mat from a Manicaria saccifera palm bract and methods of treating subjects using the biocompatible textile mesh of the present disclosure. The present disclosure also provides, engineered, biocompatible tissue constructs incorporating biocompatible textile mesh made from a naturally interwoven fiber mat from a Manicaria saccifera palm bract and a population of cells growing on the fibers of the mesh.

The field of tissue engineering, which utilizes scaffolds with complex architectures, naturally-derived materials, such as plant-based materials, are of interest for the potential to offer such properties as similar architecture, availability, low-cost, and scalability. Decellularized Spinacia oleracea (spinach) and Petroselinum crispum (parsley) leaves have been used as scaffolds to demonstrate perfusion of plant vasculature by mammalian endothelial cells and cardiomyocytes [4]. In a comparable manner, a range of cell types have been cultured on several decellularized plant stems and leaves, showing variable growth rates. Parsley promoted significant cellular growth of human mesenchymal stem cells and human dermal fibroblasts whereas Bambusoideae (bamboo) and Vanilla planifolia (vanilla) did not [5]. For the above studies it was necessary to first decellularize the plant tissues and, in most cases, to also modify the decellularized plant tissues with mammalian adhesion peptides or proteins in order to attain sufficient cell attachment and growth [6]. Biocompatibility of natural materials was demonstrated for McIntosh red apple hypanthium-derived scaffolds with immunocompetent wild-type C57BL/10ScSnJ mice by the gradual reduction of the immune response several weeks after implantation [7]. However, in one instance, in order to improve cell attachment to apple-based scaffolds extracellular matrix-based hydrogels were cast onto the scaffolds to form composite plant-derived cellulose biomaterials [8]. However, the bioactivity and low-immunogenicity of decellularized apple from this example cannot be imputed to other plant-based scaffolds, due to the high variability in properties of plant-based materials, as suggested by the minimal growth of human mesenchymal stem cells and human dermal fibroblasts on both bamboo- and vanilla-based scaffolds [5]. Due to this unpredictability, in order to consider any new plant-based materials, it is important to independently determine biocompatibility of such new material to demonstrate its relevance as a scaffold material.

The Manicaria saccifera palm, also known as Ubuçu, Tururi, bucu, monkey cap, etc. depending on the region, produces a hollow tubular bract consisting of a brown naturally woven, acellular, fibrous material with interlocked and bifurcating regions making it a fabric-like biological textile, or biotextile [9]. The fibers have a naturally interconnected/bifurcating pattern, where they naturally intertwine/interweave with each other, providing the mat of the bract with a naturally woven structure. For the palm, the bract structure acts as a sieve to protect it from insects interested in its inflorescence (flower and associated structures) [10]. The fibers forming the bract are ˜100 ˜m in diameter, are perforated by longitudinal stomata, and are composed of ˜67% cellulose and ˜31% lignin. The fiber's surface ultrastructure consists of longitudinal ridges and silica surface protrusions (5-10 μm in diameter), which when dislodged leave a crater in the fiber's surface. Bulk properties of the palm's fiber mats have been physically and mechanically characterized displaying slight variability in properties depending on where in the palm bract the mats were sourced [9]. Across a palm bract: water absorptivity ranges from ˜70-75%, volumetric density ranges from ˜0.80-1.07 g/cm³, longitudinal Young's modulus ranges from ˜1.81-2.42 GPa, longitudinal fracture stress ranges from ˜55-82 MPa, and longitudinal strain at failure ranges from ˜4.97-5.42%. Because of these properties, the fiber mats have been used as reinforcing fibers in poly(lactic acid) matrix composite materials [11,12].

With the unique surface structure of the fibers and the beneficial mechanical properties of the fiber mats, testing was performed to further investigate the mechanical properties and biocompatibility of Manicaria saccifera fibers and mats to demonstrate applicability of these naturally woven fiber mats as a minimally processed plant-based material for tissue engineering composites. The example below demonstrates the physical and mechanical properties of individual Manicaria saccifera fibers and their biocompatibility with various mammalian cell types to support attachment, growth, and survival of mammalian cells as well as their inflammatory response by peripheral blood monocytes.

Thus, the present disclosure includes, in embodiments, a naturally-derived, biocompatible textile mesh made from the naturally inter-woven fiber mats obtained from a bract of the Manicaria saccifera palm. As described in greater detail below, a significant advantage to the natural mats of the Manicaria saccifera palm is that they have a naturally occurring interwoven pattern of fiber growth, such that it has the construction of a woven textile without having to separate and weave fibers. The bract is a pendulous sac that holds the palm seeds/fruit that is made of the interwoven mesh of fibers. The bracts are sac-like and can be cut from the tree and cut open to provide a woven fiber mat. The fiber mat is naturally a-cellular so it does not need to be first decellularized like other plant-derived scaffolding materials. The fiber mat can be minimally treated by washing and a mild treatment (e.g., alkaline wash, acid wash, alternating alkaline and/or acid wash) to remove debris, natural oils, and lignin that occurs on the surface of the fibers (as opposed to the lignin that makes up the fiber itself). The lignin and oil coating provide a hydrophobic barrier to help protect the fibers from weathering, etc., but can be easily removed by a mild, non-toxic treatment to improve the hydrophilicity/wettability of the fibers. In embodiments, the mats can be treated with an alkali treatment such as described in the examples below.

In embodiments, the mats can be further treated to sterilize the mats for biological uses. In embodiments, the mats can be sterilized by autoclaving (though other methods as known to those of skill in the art may be used). In embodiments the mats are autoclaved at an appropriate temperature and time to sterilize the mats without substantial damage to the fibers. In embodiments, the mats are autoclaved at a temperature of about 105 to about 135 degrees C. for about 15 minutes or more (e.g. about 120 degrees for about 20 min).

In embodiments, the mats can be used both in vitro and in vivo (e.g., as tissue scaffolds, surgical mesh, etc.) without further processing. In other words, unlike some other plant-derived scaffolds discussed above, the Manicaria saccifera palm fiber mats do not require functionalization (e.g., with additional proteins that facilitate cell interactions, such as, but not limited to fibronectin, collagen, other extracellular matrix proteins, adhesion ligands, etc.) in order to improve biocompatibility and amenable to cell attachment. As shown in the example below, a simple wash with water and alkali treatment (e.g., sodium hypochlorite, NaClO) was all that was needed in order to successfully grow various mammalian cell types on Manicaria saccifera palm fibers in vitro. No further protein functionalization or other bio-functionalization was needed for substantial cell attachment and growth. Thus, in embodiments, a Manicaria saccifera fiber mat of the present disclosure that has been minimally treated by washing and a mild treatment to remove debris, natural oils, and lignin that occurs on the surface of the fibers (as described above) and that has not been functionalized with any additional proteins can be used to provide a biocompatible textile mesh of the present disclosure. Such minimally treated Manicaria saccifera palm fiber mats of the present disclosure can be cut to provide a naturally-derived, biocompatible, textile mesh that can be used as a biotextile in various medical and tissue engineering applications, such as, but not limited to, surgical mesh, sutures, ligament, tissue scaffolds, wound patches, and the like.

Embodiments of the present disclosure also include methods of growing mammalian cells and/or mammalian tissue using the biocompatible textile mesh of the present disclosure. In embodiments, such methods can include, providing a biocompatible textile mesh made from an interwoven fiber mat from a Manicaria saccifera palm bract such as described above, where the fiber mat has been treated to remove oils and lignin from the surface of palm fibers in the mat. In embodiments the fiber mat has been to remove oils and lignin by washing with water and an alkali solution to reduce hydrophobicity of the fibers. The method then includes seeding the biocompatible textile mesh with a population of mammalian cells, and growing the cells on the mesh to form a tissue. In embodiments, the biocompatible textile mesh consists essentially of a portion of an interwoven fiber mat cut from a bract of Manicaria saccifera that has been treated with water and alkali and has not had any further functionalization or additives prior to cell seeding. In embodiments, the biocompatible textile mesh does not comprise added extracellular matrix proteins, such as collagen, fibronectin, or added adhesion ligands.

In embodiments, growing the cells involves culturing the seeded mesh in a compatible culture medium in vitro such that the cells grow on the mesh to form a tissue. In embodiments, this can provide a tissue construct that can be used in a patient in vivo, e.g., as a tissue scaffold, tissue patch, wound patch, etc. In some embodiments, growing the cells involves implanting or applying the seeded biocompatible textile mesh in or on a subject and growing the cells to form a tissue in vivo in the subject. In embodiments, methods of growing cells/tissue of the present disclosure can include a combination of in vitro and in vivo cell growth. For instance, the biocompatible textile mesh from Manicaria saccifera can be seeded with cells, and the cells can be grown for a while in culture in vitro, and then the textile mesh with growing cells/tissue can be implanted in a subject (or applied to a tissue of a subject) for continued cell growth in vivo.

In embodiments, methods of the present disclosure also include growing mammalian tissue by providing a biocompatible textile mesh made from an interwoven fiber mat from a Manicaria saccifera palm bract as described above and implanting the biocompatible textile mesh in a mammalian subject or applying the biocompatible textile mesh to a tissue of a mammalian subject, such that the subject's own cells grow on the mesh in vivo to form a tissue. Thus, in some embodiments, the biocompatible textile may not be pre-seeded with cells, but may be implanted (or applied to a tissue of a mammalian subject, e.g., as a bandage/wound patch), such that the subject's own cells grow in/on the mesh to form a tissue construct.

Methods of the present disclosure also include methods of treating a subject by providing a biocompatible textile mesh made from an interwoven fiber mat from a Manicaria saccifera palm bract, that has been treated to remove oils and lignin from the surface of palm fibers in the mat; and implanting the biocompatible mesh in the subject such that the subject's own cells grow on the mesh. In embodiments, the subject is in need of treatment for a hernia, and the biocompatible mesh provides a hernia mesh. The biocompatible mesh of the present disclosure can also be used as other forms of surgical mesh for treating subjects in need thereof. In embodiments, the biocompatible textile mesh is used as a tissue scaffold for in-growth of the patient's own cells.

In embodiments, the mesh provides a tissue scaffold/construct pre-seeded with mammalian cells. In embodiments, the seeded cells are the patient's own cells (e.g., harvested autologous cells of the desired tissue type or of a stem cell or other pluripotent cell capable of differentiating into a desired tissue type). In embodiments, the seeded cells are human adipose-derived mesenchymal stem cells. In some instances, the patient's own cells may not be available or able to be used, and in such cases, the seeded cells can be, but are not limited to heterologous cells (e.g., heterologous stem cells from a donor or more than one donor), allogenic cells/stem cells (e.g., stem cells from a matched donor), and other compatible mammalian cells.

In embodiments, the biocompatible textile mesh can be used as a wound patch in treatment of a patient with a wound. In embodiments, the biocompatible textile mesh of the present disclosure may be used as a tissue patch to treat a patient in need of a graft or tissue scaffold.

Embodiments of the present disclosure also include engineered, biocompatible tissue constructs including the biocompatible mesh of the present disclosure. In embodiments, such tissue constructs can include a biocompatible textile mesh made from an interwoven fiber mat from a Manicaria saccifera palm bract, wherein the fiber mat has been treated to remove oils and lignin from the surface of palm fibers in the mat; and a population of living cells growing on the fibers of the mesh. In embodiments, the cells are mammalian cells, such as, but not limited to human cells, mouse cells, mammalian stem cells, etc. In embodiments the cells are human epithelial cells. In embodiments, the cells have grown on the mesh sufficient to form a tissue layer. In embodiments, the population of cells growing on the tissue construct include more than one cell type.

Additional details regarding the tests and methods of the present disclosure are provided in the Examples below. The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and protected by the following embodiments.

The following examples are put forth so as to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value.

### EXAMPLE

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Introduction

Plant-based fibers are a potential alternative to synthetic polymer fibers that can yield enhanced biocompatibility and mechanical properties matching those properties of tissue. Given the unique morphology of the bract of the Manicaria saccifera palm, being an interwoven meshwork of fibers, these fibers with this built-in structure could prove useful as a tissue engineering scaffold material. Thus, in the present example, the fiber's in vitro biocompatibility and immunogenicity is first investigated. NIH/3T3 mouse fibroblasts, human aortic smooth muscle cells, and human adipose-derived mesenchymal stem cells were cultured on the fiber mats, which all readily attached and over 21 days grew to engulf the fibers. Importantly, this was achieved without treating the plant tissue with extracellular matrix proteins or any adhesion ligands. In addition, the gene expression and protein secretion of three target inflammatory cytokines (IL-1β, IL-8, and TNFα) from THP-1 human leukemia monocytes cultured in the presence of the biotextile as an in vitro immunological model were measured. After 24 hours of culture, gene expression and protein secretion were largely the same as the control, demonstrating the low immunogenicity of Manicaria saccifera fibers. The tensile mechanical properties of the fibers were also measured. Individual fibers after processing had a Young's modulus of 9.51±4.38 GPa and a tensile strength of 68.62±27.93 MPa. The tensile mechanical properties of the fiber mats perpendicular to the fiber axis (transverse loading) were investigated, which displayed upwards of 100% strain, but with a concession in strength compared to longitudinal loading. Collectively, the in vitro assessments point towards Manicaria saccifera as a highly biocompatible biotextile, with a range of potential clinical and engineering applications.

### Materials & Methods

Materials

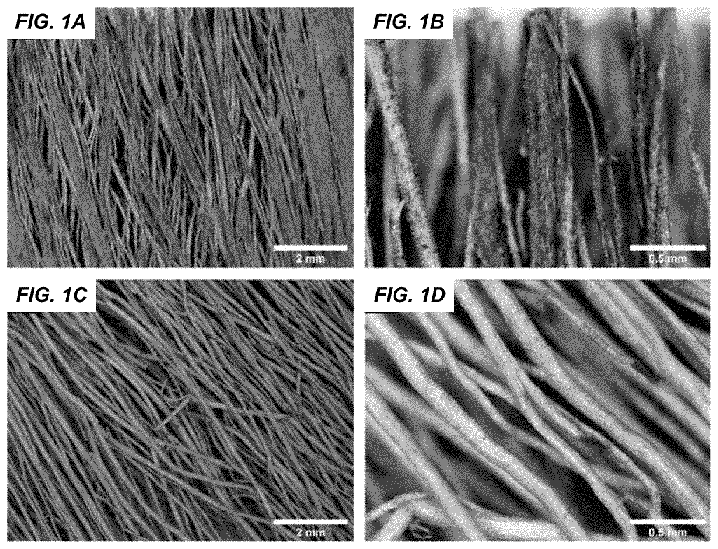

Manicaria saccifera bracts were manually collected by a native community in the Chocó region in Colombia and obtained by A. Marañon at Universidad de los Andes in Colombia as previously described [9]. Native Manicaria saccifera bracts vary in length, but sleeve strips 60 to 120 cm long can be typically harvested. For cell culture, the fiber mats were washed, and alkali treated to strip away lignin and other surface impurities, and improve their wettability (see Porras, et al., 2016, reference 12, incorporated herein by reference). Representative images of the fibers before and after alkali treatment is presented in FIG. 1. For both mechanical and biocompatibility tests, alkali treated Manicaria saccifera individual fibers and fiber mats were sterilized by steam autoclaving at 121° C. for 20 minutes.

Shown in FIG. 1 are Manicaria saccifera fiber mats before (A, B) and after (C, D) alkali treating. The treatment process is intended to strip away lignin and other surface impurities. Following treatment, the surface of the fibers appeared smoother and fiber color faded.

Tensile Testing

Although the mechanical properties of Manicaria saccifera bracts have been previously reported, here the focus is on the mechanical properties of individual fibers both with and without treatment [9,12,13]. In addition, the mechanical properties of the fiber mat in the transverse direction (i.e., perpendicular to fiber axial direction) were also measured primarily because these have not been extensively reported in literature and to gain further insight into the anisotropic behavior of the mat. Samples were tested using a Stable Micro Systems TA.XT Plus Texture Analyzer. Individual fiber samples from untreated, alkali treated, and alkali-treated and autoclaved conditions were analyzed. A minimum of 10 samples per condition were measured. Fiber diameters were individually measured with diameters varying between 250-350 μm and cut into specimens with average length of 4.56 cm and then weighed to calculate the individual count number as described below. Consistent with previously reported values, 0.97 specific gravity was used for the fibers [14]. To secure the fibers in the tensile testing grips, the fibers were embedded in polydimethylsiloxane elastomer or secured in masking tape. No apparent difference in the load-displacement curves was found between the two methods for securing the fibers. All specimens were tested to failure at strain rates of 0.17 mm/s (10 mm/min). The effective stress was calculated from tenacity and specific gravity of the fibers using γ=F/Tₘ (1) and σ=10gₛₚγ (2), where γ is tenacity (cN/tex), F is the load (cN), Tₘ is the count number (tex=g/km), σ is stress (MPa) and gₛₚ is specific gravity [15]. The Young's modulus was estimated from the slope of the initial linear region of each stress-strain curve. Tensile strength was taken as the stress at failure.

Cell Culture

Four different cell types were cultured with the Manicaria saccifera fibers, NIH/3T3 (ATCC® CRL-1658™) (NIH3T3s), human adipose-derived mesenchymal stem cells (HADSCs) (Lifeline Cell Technologies), human aortic smooth muscle cells (HAoSMCs) (Cell Applications), and THP-1 (ATCC® TIB-202™). NIH3T3s were cultured in Dulbecco's Modified Eagle Media (Corning) media supplemented with 10% heat-inactivated fetal bovine serum (Corning), 1% penicillin/streptomycin (Corning), and 1% amphotericin-B (Corning). HADSCs were cultured in StemLife MSC complete media (Lifeline Cell Technologies). AoSMCs were cultured in human SMC complete media (Cell Applications). THP-1 monocytes were cultured in RPMI medium 1640 (Gibco and ATCC) supplemented with 10% non-heat inactivated fetal bovine serum (Corning and Gibco), 0.05 mM β-mercaptoethanol (Bio-Rad and Gibco), 1% penicillin/streptomycin (Corning), and 0.02% amphotericin-B (Corning). All cells were cultured in a humidified incubator with 5% CO₂ at 37° C. In all cases, the media was changed every 2-3 days.

Cell Attachment, Viability, Proliferation, and Tissue Formation

Alkali treated and autoclaved Manicaria saccifera fiber mats were cut into small samples of ˜4×4×1 mm. These samples were then placed into individual wells of a 24-well ultra-low attachment well plate (Corning). NIH3T3s, HADSCs, and HAoSMCs were seeded onto the Manicaria saccifera in their respective growth media at 50,000 cells/well. After 48 hours, the growth media was removed, fibers washed twice with phosphate-buffered saline (PBS) (Corning) to remove unattached cells, and then fresh media was added to each well. The media was changed every 2-3 days. Images were taken over 21 days to qualitatively visualize cellular attachment and proliferation on the Manicaria saccifera fiber mats. At 21 days, staining for viable cells bound to the fiber mats was performed using a Pierce LIVE/DEAD™ Viability/Cytotoxicity Kit, for mammalian cells (Invitrogen) following the manufacturer's protocol. In this assay, live cells stain with green fluorescence from the intracellular uptake of calcein AM, while dead and dying cells stain with red fluorescence from binding of ethidium homodimer-1 to exposed nuclear DNA. The cells attached to the Manicaria saccifera were then imaged by fluorescence microscopy. All cell culture imaging was conducted using a Nikon TE2000-U inverted phase contrast/epifluorescence microscope. To assess tissue formation and matrix production, a subset of fiber mats cultured with each cell type were histologically stained and sectioned. The fiber mats were dehydrated in graded ethanol, embedded into paraffin blocks, sectioned as 7 μm slices using a Microm HM355S rotary microtome (Thermo Scientific), and mounted onto glass slides. The sections were then deparaffinized, rehydrated, and stained with hematoxylin and eosin to visualize cells bound to Manicaria saccifera fiber mats as well as any extracellular collagen. Stained slide sections were imaged using a Nikon TE2000-U inverted microscope. Fiber ultrastructure after cell culture was imaged by scanning electron microscopy (SEM). Samples were prepared by mounting onto microscopy stubs and coating with gold-palladium or carbon to reduce sample charging. Samples were imaged using a PhenomWorld ProX desktop electron microscope (PhenomWorld) at a beam energy of 10-15 kV.

In Vitro Immunogenicity Assay

In vitro immunogenicity was assessed by measuring THP-1 monocyte inflammatory gene expression and inflammatory cytokine secretion after culture in the presence of Manicaria saccifera fibers. THP-1 cells were seeded at 1,000,000 cells/well into a 24-well ultra-low attachment well plate in their complete growth medium. To assess their inflammatory response to the Manicaria saccifera fibers, a sample of the fiber mat (4×4×1 mm) was added into the well. The addition of 100 μL of PBS and 100 μL of 50 μg/mL lipopolysaccharide (LPS) to separate subcultures were used as negative and positive controls for inflammatory response, respectively. Each condition was measured in triplicate. Following the addition of the materials and reagents, the THP-1 monocytes were incubated for either 4 or 24 hours before being collected for gene expression analysis. At 24 hours, cell culture media was collected for measuring inflammatory cytokine secretion. The gene expression of three inflammatory cytokine genes in THP-1 monocytes was measured by reverse transcription quantitative polymerase chain reaction (RT-qPCR). Briefly, at 4 and 24 hours the THP-1 monocytes were collected and pelleted by centrifugation. Then RNA was isolated and purified for each treatment using a RNeasy Plus Mini Kit (Qiagen) following the manufacturers protocol. Complimentary DNA for each sample was synthesized using an iScript™ cDNA Synthesis Kit (Bio-Rad). RT-qPCR was conducted with a CFX Connect™ Real-Time PCR Detection System (Bio-Rad) using iTaq™ Universal SYBR® Green Supermix (Bio-Rad) and primers from Integrated DNA Technologies for inflammatory cytokine encoding genes: interleukin-1β (IL-1β), interleukin-8 (IL-8), tumor necrosis factor-alpha (TNFα), and with housekeeping gene β2-microglobulin (B2M) serving as a reference gene. The ΔΔCq calculation method was used to determine relative gene expression. To assess inflammatory cytokine secretion, after 24 hours of culture, aliquots of cell culture media was collected and centrifuged from the different treatments. The relative concentrations of inflammatory cytokines in the supernatant for each sample were determined using a Human Inflammatory Cytokines Multi-Analyte ELISArray™ kit (Qiagen) according to the manufacturer's instructions. The inflammatory cytokines investigated by this array were IL-1β, IL-8, and TNFα.

Statistical Analysis

All statistical analyses were conducted using GraphPad Prism 8. When applicable, student's t-test or ANOVA were used to determine statistical significance between conditions using a significance value of 0.05. Multiple comparison testing was performed against controls using Dunnett's test and between treatments using Tukey's test. Data is presented as the mean and standard deviation unless otherwise noted.

Results

Manicaria saccifera Mechanical Properties

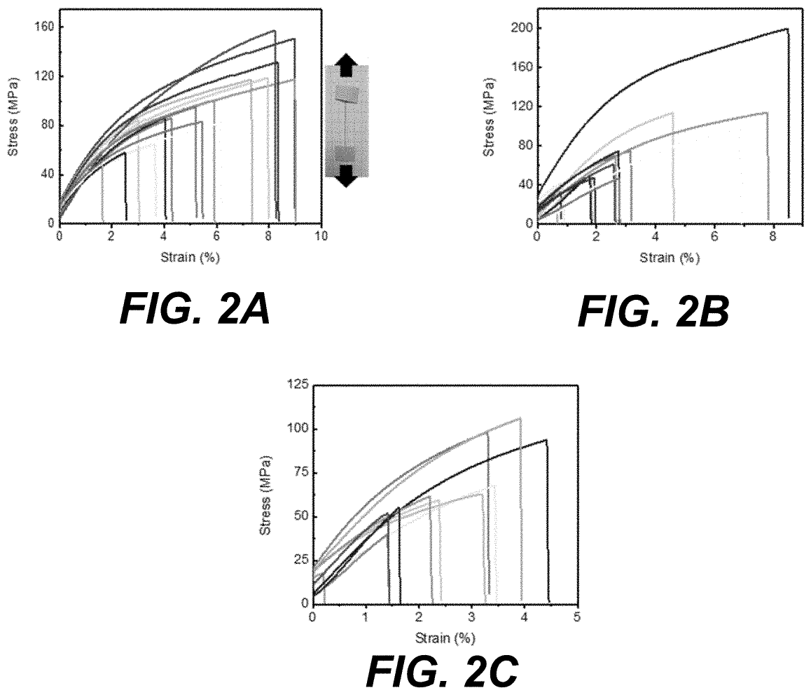

Fibers demonstrated a range of tensile behavior by either increasing linearly with increasing strain followed by a slight non-linearity just before failure or following a gradual linear increase with increasing strain until an inflection point was reached and the stress increased more rapidly with increasing strain until failure. Stress calculated from equations 1 and 2 was plotted against percent strain (FIG. 2).

Shown in FIG. 2 are results of fiber tensile testing for individual Manicaria saccifera: a) untreated fibers, b) alkali treated and c) alkali treated and autoclaved. Fibers were pulled at a strain rate of 10 mm/min (0.17 mm/s). There large degree of variability observed in the stress-strain curves is common in natural fibers.

Spikes before failure in some curves were attributed to partial failure of the cell columns within the bundles that form each fiber. It is important to note that fiber moduli and strength decreased and strain at failure increased when longer fibers were tested and when fibers were tested at a slower strain rates. Therefore, it is important to compare the results of the mechanical tests on samples with similar lengths and at the same strain rate. Table 1 presents the summary of mechanical properties measured in this work. It is important to note that the alkali and autoclave treatment led to a reduction in the tensile strength and strain at failure with a corresponding increase in the effective Young's modulus. A somewhat stiffer fiber after processing, that is also less ductile and less strong, is consistent with the stripping of oils by the alkali treatment and water reduction due to autoclaving.

TABLE 1
Summary of individual Manicaria saccifera fiber effective mechanical properties.

| | Number | | Fiber | Fiber | Strain | Young's | Tensile | Strain at |
|---|---|---|---|---|---|---|---|---|
| Type of | of | Weight | Length | Diameter | Rate | Modulus | Strength | Failure |
| fiber | Samples | (mg) | (cm) | (μm) | (mm/s) | (GPa) | (MPa) | (%) |
| Untreated | 16 | 1.51 ± 0.25 | 4.73 ± 0.18 | 289.31 ± 26.7 | 0.17 | 3.10 ± 1.04 | 99.36 ± 30.45 | 5.71 ± 2.4 |
| Alkali | 15 | 1.09 ± 0.16 | 4.4 ± 0.46 | 309.64 ± 45.6 | 0.17 | 8.22 ± 4.86 | 72.38 ± 45.19 | 3.33 ± 2.52 |
| treated | | | | | | | | |
| Alkali | 11 | 1.07 ± 0.21 | 4.56 ± 0.72 | 310.47 ± 39.55 | 0.17 | 9.51 ± 4.38 | 68.62 ± 27.93 | 2.80 ± 1.52 |
| treated | | | | | | | | |
| and | | | | | | | | |
| autoclaved | | | | | | | | |

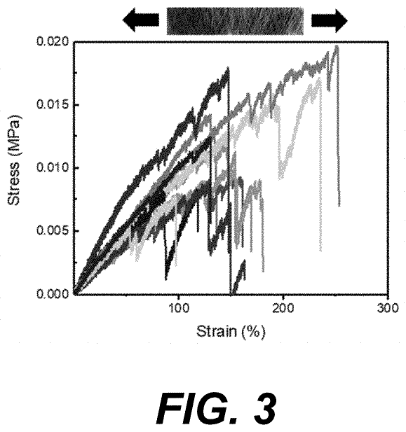

Longitudinal mechanical properties of Manicaria saccifera fiber mats were reported in Porras, et al. (2015) (reference 9, below, which is hereby incorporated by reference herein as applicable. With respect to the human body, mechanical loading is not uniform in a single direction and thus, the transverse mechanical properties of Manicaria saccifera fiber mats were also measured in the present example in order to further determine feasibility for use of the mats in a biological environment in vivo. When loaded transversally, the fiber mats were able to sustain significant elongation (>100% strain) before failure (FIG. 3). This ability was attributed to the fiber mat's lack of cross-weaving in the transverse direction. The transverse Young's modulus was 98±11 Pa and the tensile strength was 12±2 kPa. The fiber mats displayed a trade-off for increased ductility with an accompanied loss in tensile strength. Accordingly, the Young's modulus and tensile strength were found to be much lower than their longitudinal counterpart.

Shown in FIG. 3 is an example of transverse tensile loading of Manicaria saccifera fiber mats. The fiber mats displayed upwards of 100% strain; however, the mats are significantly weaker compared to longitudinal loading.

Manicaria saccifera Biocompatibility

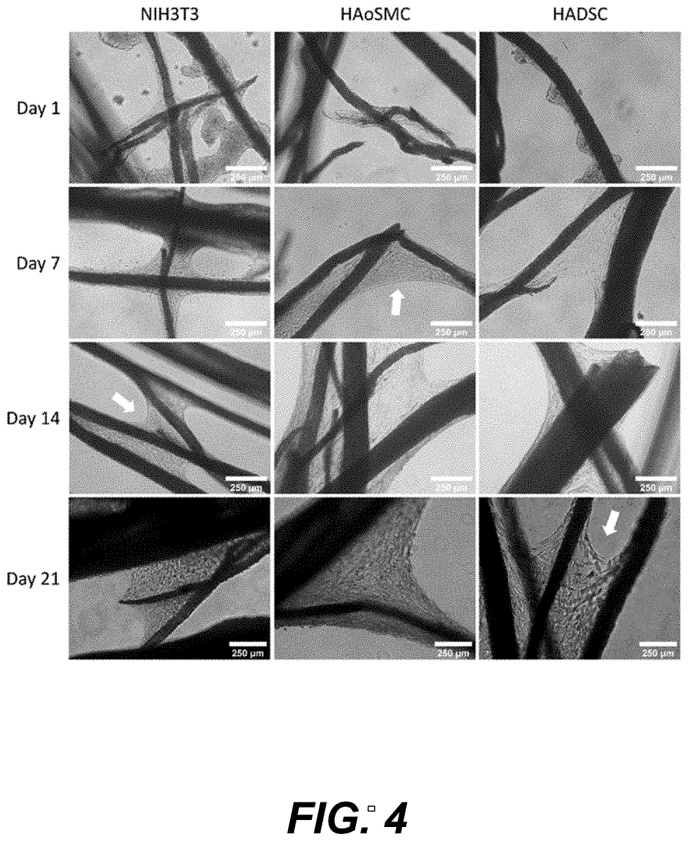

Over 21 days, cells seeded onto Manicaria saccifera fiber mats were imaged. As soon as one day after seeding, cells readily attached to the Manicaria saccifera fibers. After 7 days, cell structures were clearly defined as well as cellular organization for all cell types (FIG. 4). The HADSCs appeared to be the least densely organized while HAoSMCs and NIH3T3s grew and engulfed the individual fibers composing the fiber mat. There was significant contact guided alignment of cells along the fiber longitudinal axis. Qualitatively, in all cases, each cell type continued to proliferate through 21-days. In addition, the formation of cellular bridges between proximal fibers was observed, which were at times greater than 500 μm in distance.

Shown in FIG. 4 is an example of the extended culture of the different cell types on Manicaria saccifera fiber mats. NIH3T3s, HAoSMCs, and HADSCs were all able to attach to the fibers after 1 day. Over a period of 3 weeks, the cells engulfed the fibers and formed webbings between proximal fibers examples of which are indicated by white arrows.

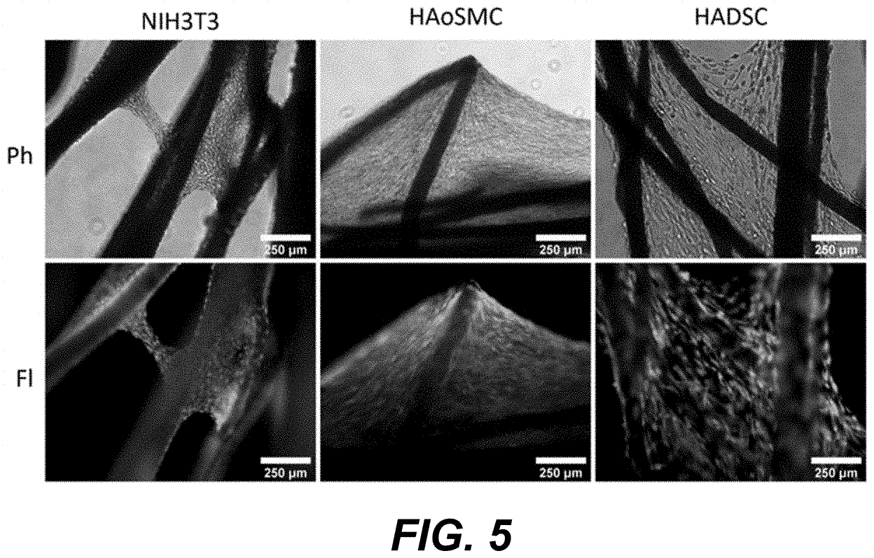

Cell viability after extended culture was assessed using a Pierce™ Live/Dead Viability/Cytotoxicity assay kit. After 21 days, an abundance of cells stained green from the live cell label, calcein AM. Conversely, there was an absence of red ethidium homodimer-1 fluorescence for each cell type indicating there was minimal cell death for cells growing on Manicaria saccifera fibers (FIG. 5).

Shown in FIG. 5 an example of the cell viability on Manicaria saccifera fiber mats. At 21 days, each cell type showed robust cellular viability at multiple levels of thickness indicated by the strong green fluorescence from the calcein AM live cell staining. There were little to no dying or dead cells indicated by the absence of red ethidium homodimer-1 staining in the fluorescence (FI) images. Phase contrast (Ph) images are shown to indicate the location of the fibers.

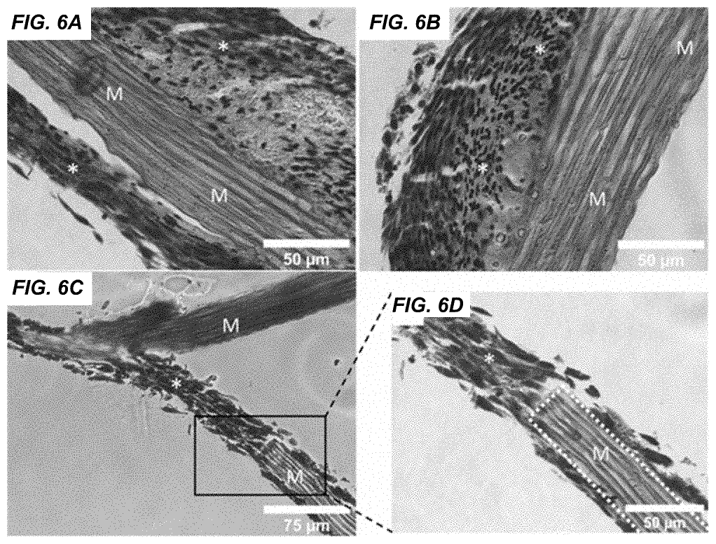

Histological assessments were also conducted to better visualize the interaction between the cells and the fibers. Histological staining revealed and confirmed robust cellular attachment and proliferation onto the Manicaria saccifera fibers (FIG. 6). In addition, it further exemplified an observed contact guidance supported the fibers, which directed cellular alignment several layers thick to be parallel to the fiber longitudinal axis. Multiple layers of cells along with extracellular matrix collagen was seen surrounding the fibers. Interestingly, bridging between individual fibers seen by phase contrast microscopy (FIG. 5) could be viewed histologically (FIGS. 6C and 6D).

Shown in FIG. 6 are examples of histological analysis of NIH/3T3 mouse fibroblasts cultured on Manicaria saccifera fiber mats. Sections showed cells aligned along the fiber axis and this alignment extended over several layers of cells (A). Additionally, some regions showed masses of randomly aligned cells surrounded by a layer of several cells aligned with the fiber axis (A, B). Cellular bridging across fibers was also observed (C, D). M=Manicaria saccifera fiber, *=NIH/3T3 mouse fibroblasts.

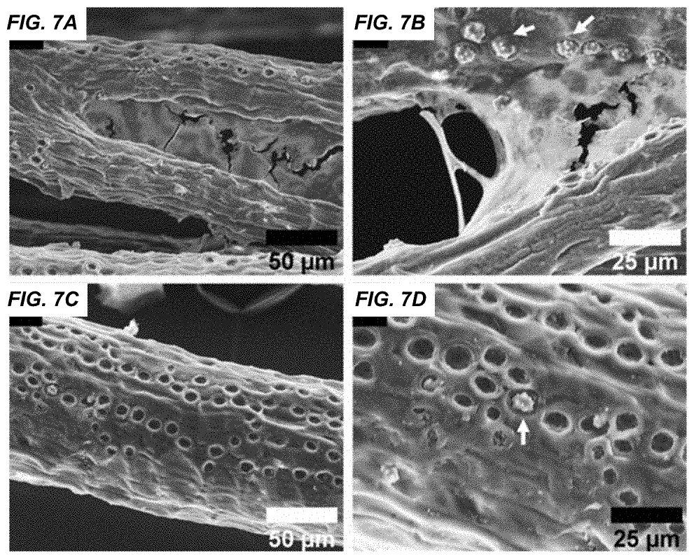

Scanning electron microscopy also confirmed cellular attachment for all observed cell types. We observed the robust cellular bridging of NIH3T3s between fibers (FIGS. 7A and 7B). We also saw fiber encapsulation by HADSC (FIG. 7C). In addition, remaining silicate deposits were seen embedded in the circular depressions along the fiber length (FIGS. 7B and 7D, arrows). Manicaria saccifera fiber mats promoted contact guided growth and proliferation of mouse fibroblasts, vascular smooth muscle cells, and mesenchymal stem cells over an extended period of culture.

Shown in FIG. 7, examples of SEM micrographs of NIH3T3s (A, B) and HADSC (C, D) on Manicaria saccifera fibers. Bridging across fibers was observed by SEM (b). Fiber dimples with and without silicate particles were observed marked by arrows (B, D)

Manicaria saccifera Immunogenicity

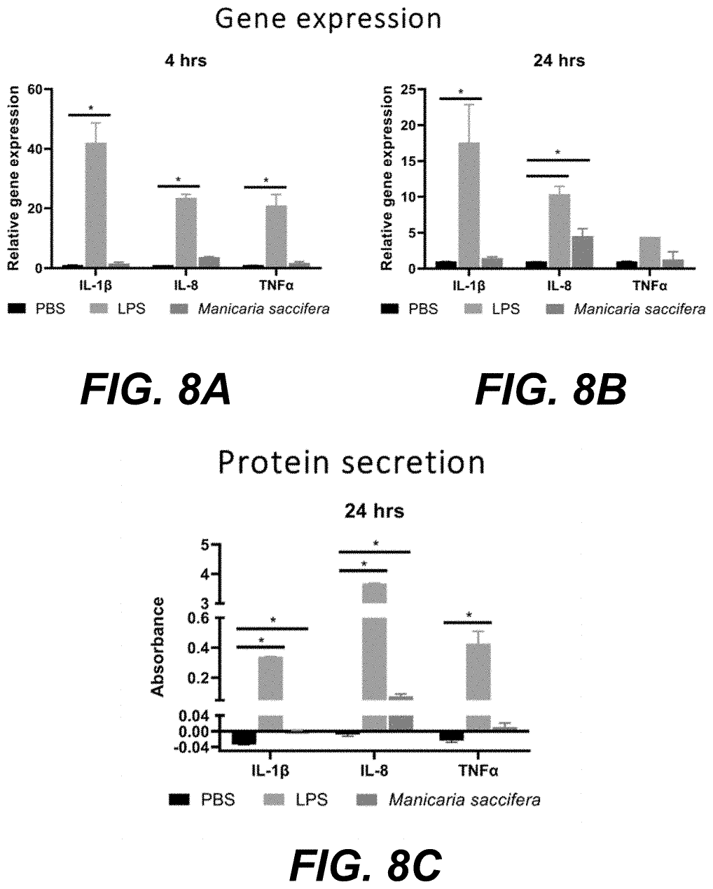

In vitro immunological response was assessed at both the gene and protein expression levels for THP-1 human leukemia monocytes. Gene expression: after 4 hours in the presence of Manicaria saccifera fiber mats, there was no significant difference in gene expression level for IL-1β, IL-8, or TNFα inflammatory cytokines compared to PBS whereas after 4 hours following LPS treatment there was a significant upregulation in gene expression of all three inflammatory cytokines. With LPS treatment at 4 hours, IL-1β showed a ˜40-fold upregulation while IL-8 and TNFα were upregulated ˜20-fold (FIG. 8A). After 24 hours in the presence of Manicaria saccifera fiber mats, there was a significant upregulation in gene expression only for IL-8 compared to PBS whereas after 24 hours following LPS treatment there was no longer significant upregulation in gene expression of TNFα. At 24 hours following LPS treatment, IL-1β showed a ˜15-fold upregulation, IL-8 showed a ˜10-fold upregulation, and TNFα showed a ˜5-fold upregulation FIG. 8B). THP-1 monocytes in the presence of Manicaria saccifera fiber mats only showed significant upregulation in IL-8 after 24 hours, with a ˜5-fold increase (FIGS. 8A and 8B). Protein expression: after 24 hours the protein expression agreed well with the gene expression results, realizing the low immunogenicity of Manicaria saccifera fibers. There was a statistically significant increase in secretion of every inflammatory cytokine following LPS treatment. After 24 hours, THP-1 monocyte secretion of inflammatory cytokines in the presence of Manicaria saccifera fiber mats corresponded to a measured absorbance of 0.0017±0.0015 (n=3) for IL-1β, 0.08±0.03 (n=3) for IL-8, and 0.011±0.018 (n=3) for TNFα (FIG. 8C). In comparison, 24 hours following LPS treatment corresponded to a measured absorbance of 0.340±0.004 (n=3) for IL-1β, 3.678±0.009 (n=2) for IL-8, and 0.42±0.14 (n=3) for TNFα (FIG. 8C).

The above values equate to 200 times more IL-1β, 46 times more IL-8, and 38 times more TNFα being secreted after 24 hours due to the LPS treatment as compared to the Manicaria saccifera fiber mat treatment. Despite a statistically significant difference for secreted IL-1β and IL-8 in the Manicaria saccifera treatment as compared to PBS, these values were drastically smaller than those induced by the LPS treatment. Thus, the above in vitro immunological assessment demonstrated that there was little to no genetic immunological response by THP-1 cells when cultured in the presence of Manicaria saccifera fiber mats.

Shown in FIG. 8 are examples of in vitro immunological assessment of Manicaria saccifera fiber mats. Gene expression after 4 hrs (a) and 24 hrs (b) and protein expression after 24 hrs (c) of interleukin-1β (IL-1β), interleukin-8, (IL-8), and tumor necrosis factor α (TNFα). Statistical significance between treatments is denoted by *. Treatments were measured in triplicate.

Discussion

This example demonstrates the identification of a promising natural biotextile derived from the Ubuçu palm, Manicaria saccifera. The first step to better understand the potential of this material in biological and engineering applications was to demonstrate its in vitro biocompatibility. The above data and results showed that Manicaria saccifera fiber mats promoted mouse fibroblast, human vascular smooth muscle cell, and human adipose-derived mesenchymal stem cell growth over an extended culture period of 21 days. Moreover, these cell types readily attached and encapsulated the fibers without any modification of the fibers by native extracellular matrix proteins. Others have tested the attachment of human mesenchymal stem cells, human dermal fibroblasts, human umbilical vein endothelial cells, human embryonic stem cell derived cardiomyocytes, C2C12 mouse myoblasts, NIH3T3 mouse fibroblasts, HeLa human epithelial cells, and MCF-7 human breast cancer cells to a number of decellularized plants, but have only demonstrated success after treating the plant materials with native extracellular matrix proteins (collagen, gelatin, fibronectin) [4-8,16]. In the case of Manicaria saccifera, such treatment was unnecessary for successful cell attachment to the material, which further reduces the amount of processing and expense required for using this biotextile in a biomedical setting. The fiber mats of the present example were only alkali treated and autoclaved prior to cell culture; no additional treatment and/or functionalization was required prior to seeding.

Alkali treatment of palm fibers has been shown to increase their number of reactive hydroxyl groups [17]. This process likely works to improve cell attachment in the same way that plasma treatment does for polystyrene, which yields a more hydrophilic, negatively charged surface [18]. In a similar manner, NIH3T3 fibroblast growth and attachment to hydrogels produced using agave fibers demonstrated a dependence on sodium hypochlorite treatment conditions [19]. When treated with greater concentrations of sodium hypochlorite, the agave fibers were shown to have diminished lignin and increased hydroxyl group content, as was the case for alkali treated palm fibers. Interestingly, reports of decellularized plant tissue, which used sodium chlorite in their processing required extracellular matrix proteins to promote effective attachment [4,5]. Additionally, in the case of decellularized plant tissue, the need to use extracellular matrix proteins to promote effective attachment could be due to remaining lignin following the decellularization treatment whereas an alkali treatment strips the lignin from the fibers [4,5]. Lignin imparts hydrophobicity to cell walls and thus residual lignin at the surface could interfere with cell attachment [5,18,20]. Further chemical analysis of plant tissue after various treatments can be undertaken to further improve cell attachment to natural materials. The present results with Manicaria saccifera demonstrate robust cell attachment and proliferation among a variety of divergent cell types.

The attachment data and the near 100% cell viability on the fibers after 21 days of culture supports the notion that these fibers are non-cytotoxic. Other studies have shown little to no cytotoxicity for woven natural fibers derived from the ramie plant (Boehmeria nivea) and for soy fibers [16,21]. These assessments corroborate the present findings that biotextiles pose little cytotoxic effects to cells cultured in their presence; however, neither of these studies showed attachment and growth of cells on these fibers. The present data demonstrated contact-guided cellular alignment with the fiber longitudinal axis, cellular engulfment of the fibers, and cellular bridging between fibers spaced many cell lengths apart. To date, few plant-based materials with intact plant architecture have been investigated for their immunological response. A suture made from woven ramie fibers showed low to mild elevation of IL-1β and TNFα in rats following implantation [16]. Apple-based cellulose scaffolds also have shown little to no immune response in mice at late time points after implantation [7]. However, as discussed above, there is wide variability between properties of plant-derived scaffold material and their ability to support cell growth and attachment. To further demonstrate biocompatibility of Manicaria saccifera, the immunological response of THP-1 human leukemia monocytes cultured in the presence of Manicaria saccifera fiber mats was investigated. From both gene expression and protein secretion of target inflammatory cytokines, it was concluded that the fibers posed little in vitro immunogenic response; though, there was a noticed upregulation of IL-8 gene expression and elevated protein secretion of IL-8 by THP-1 monocytes after 24 hours. Future in vivo investigations can be conducted to elucidate this response as a lasting effect as other plant-based scaffolds have recovered from an initial severe immune response a few weeks following implantation [7,22]. The in vitro assessments of the present example demonstrate Manicaria saccifera as a highly biocompatible biotextile.

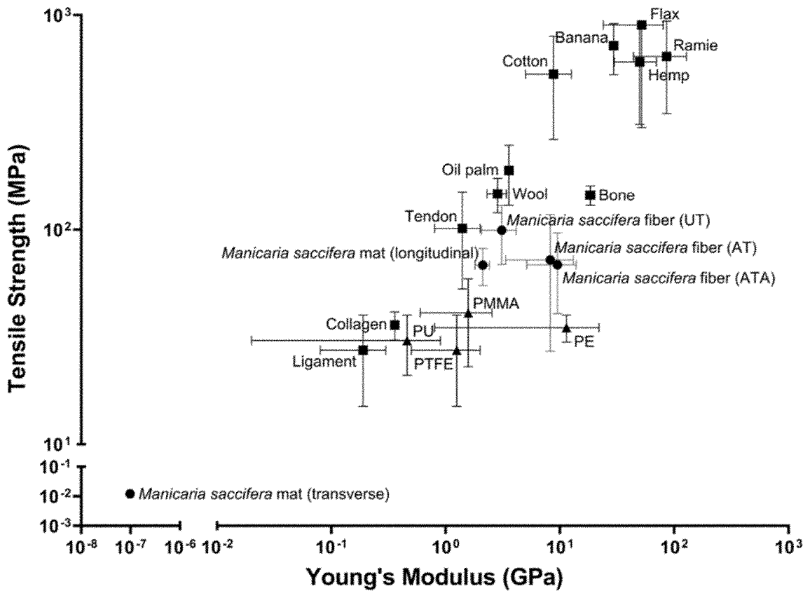

When considering this natural material for biomedical applications, investigation of the mechanical properties of both the individual fibers as well as the fiber mat were conducted. Previous reports characterized the physical, chemical, and longitudinal tensile properties of Manicaria saccifera fiber mats and proposed its use in polymer fiber reinforced composites, which were demonstrated by making a poly-lactic acid composite [9,11,12]. Because the fiber mats have an anisotropic fiber orientation, their transverse mechanical properties were qualified. The experiments above demonstrated that the fiber mats were able to sustain greater than 100% strain when loaded in the transverse direction; however, the tensile strength was reduced compared to longitudinal loading. Especially important to biomedical applications are the mechanical properties of the individual fibers because these are the properties sensed at the cellular level [23]. The above data demonstrated that, depending on orientation and loading conditions, the fibers are uniquely positioned being stiffer and stronger than common engineering polymers (polyethylene, polyurethane, polymethylmethacrylate, polytetrafluorethylene), while being softer and weaker than other traditional natural fibers (hemp, flax, ramie, cotton) (FIG. 9). It was of note that both individual fibers and the fiber mat of Manicaria saccifera offer elasticity and strength values that are close to that of tendon (FIG. 9). Incorporation of these fibers in a biocompatible composite could prove valuable in achieving modest mechanical property optimization for these target tissues. Importantly, Manicaria saccifera fibers are robust and durable. Their mechanical properties showed little change upon alkali treatment and sterilization by autoclaving. These beneficial processing properties and the fibers' extended shelf-life (stored dry at room-temperature indefinitely) further enable their applicability to the clinic where sterilizability and off-the-shelf devices are desired.

Shown in FIG. 9 is an Ashby plot of natural (▪) and common synthetic (▴) materials for tissue engineering. Each material is plotted as a function of the tensile strength and Young's modulus (stiffness). The mechanical properties of Manicaria saccifera individual fibers and fiber mats (●) are highlighted. The transverse fiber mat mechanical properties are shown using a segmented x and y axis in the figure. UT=untreated, AT=alkali treated, ATA=alkali treated and autoclaved, PU=polyurethane, PTFE=polytetrafluoroethylene, PMMA=polymethylmethacrylate, PE=polyethylene. Data compiled was from [9,27-30].

Because of its mechanical properties and biocompatibility, a Manicaria saccifera biotextile has the potential to be used for many biomedical applications, such as hernia meshes and scaffolds for tendon tissue engineering. Hernia meshes require material properties similar to the abdominal wall (tensile strength ˜16 N) and macroporous architecture (pores>2.5 mm) for tissue in-growth and lower foreign body response [24]. Manicaria saccifera mechanical properties fair well (˜5 N for a single fiber) such that as a composite material these properties can be optimized to match those of the abdominal wall. Given built-in interwoven meshwork of the fiber mats and its biocompatibility suggest its usefulness as a hernia mesh. Likewise, tendon tissue engineering scaffolds require material properties similar to the native tissue (tensile strength ˜50 MPa, Young's modulus ˜1.1 GPa), porosity (200-250 μm pores), woven architecture, and biocompatibility [25,26]. Manicaria saccifera fibers satisfy many of these properties being of comparable tensile strength (˜68 MPa) and pore size; though, the fibers are somewhat stiffer (˜9×) than native tendon. Especially beneficial is the demonstrated biocompatibility of adipose-derived mesenchymal stem cells on these fibers being a desirable cell source for tissue engineering applications. Given the biotextile's mechanical and biocompatible properties, further work is necessary to investigate Manicaria saccifera fibers for these promising potential applications.

### Conclusions

Because of its mechanical properties and biocompatibility, a Manicaria saccifera biotextile has the potential to be used for many biomedical applications including, but not limited to: hernia meshes and scaffolds for tendon tissue engineering. Matching mechanical properties to surrounding tissue is paramount for biomedical devices; thus, Manicaria saccifera fibers are unique in that they have mechanics remarkably similar to collagenous fibers unlike other natural fibers and engineered plastics [26,28,30-32]. Moreover, these fibers can be easily stored and sterilized by steam autoclaving. Paired with the biotextile's demonstrated in vitro biocompatibility, makes it potentially competitive with current hernia mesh and tissue-engineered tendon biomaterials [24,26]. Additionally, Manicaria saccifera fiber mats may be used with other materials to form composites further tailoring and expanding the potential applications of this biocompatible biotextile [12]. Plant-based composites are gaining notoriety as next-generation biomaterials and Manicaria saccifera promises to expand their utility [27,28].

### REFERENCES

[1] M. I. Misnon, M. M. Islam, J. A. Epaarachchi, K. Lau, Potentiality of utilising natural textile materials for engineering composites applications, Mater. Des. 59 (2014) 359-368. doi:10.1016/j.matdes.2014.03.022.

[2] U. G. K. Wegst, H. Bai, E. Saiz, A. P. Tomsia, R. O. Ritchie, Bioinspired structural materials, Nat. Mater. 14 (2015) 23-36. doi:10.1038/nmat4089.

[3] R. Mohammadinejad, S. Karimi, S. Iravani, R. S. Varma, Plant-derived nanostructures:

types and applications, Green Chem. 18 (2016) 20-52. doi:10.1039/C5GC01403D.

[4] J. R. Gershlak, S. Hernandez, G. Fontana, L. R. Perreault, K. J. Hansen, S. A. Larson, B. Y. K. Binder, D. M. Dolivo, T. Yang, T. Dominko, M. W. Rolle, P. J. Weathers, F. Medina-Bolivar, C. L. Cramer, W. L. Murphy, G. R. Gaudette, Crossing kingdoms: Using decellularized plants as perfusable tissue engineering scaffolds, Biomaterials. 125 (2017) 13-22. doi:10.1016/j.biomaterials.2017.02.011.

[5] G. Fontana, J. Gershlak, M. Adamski, J.-S. Lee, S. Matsumoto, H. D. Le, B. Binder, J. Wirth, G. Gaudette, W. L. Murphy, Biofunctionalized Plants as Diverse Biomaterials for Human Cell Culture, Adv. Healthc. Mater. 6 (2017) 1601225. doi:10.1002/adhm.201601225.

[6] D. J. Modulevsky, C. Lefebvre, K. Haase, Z. Al-Rekabi, A. E. Pelling, Apple Derived Cellulose Scaffolds for 3D Mammalian Cell Culture, PLoS One. 9 (2014) e97835. doi:10.1371/journal.pone.0097835.

[7] D. J. Modulevsky, C. M. Cuerrier, A. E. Pelling, Biocompatibility of Subcutaneously Implanted Plant-Derived Cellulose Biomaterials., PLoS One. 11 (2016) e0157894. doi:10.1371/journal.pone.0157894.

[8] R. J. Hickey, D. J. Modulevsky, C. M. Cuerrier, A. E. Pelling, Customizing the Shape and Microenvironment Biochemistry of Biocompatible Macroscopic Plant-Derived Cellulose Scaffolds, ACS Biomater. Sci. Eng. 4 (2018) 3726-3736. doi:10.1021/acsbiomaterials.8b00178.

[9] A. Porras, A. Maranon, I. A. Ashcroft, Characterization of a novel natural cellulose fabric from Manicaria saccifera palm as possible reinforcement of composite materials, Compos. Part B Eng. 74 (2015) 66-73. doi:10.1016/j.compositesb.2014.12.033.

[10] J. C. Copete, D. M. Flórez, L. A. Núñez-Avellaneda, Pollination Ecology of the Manicaria saccifera (ARECACEAE): A Rare Case of Pollinator Exclusion, in: Pollinat. Plants, InTech, 2018. doi:10.5772/intechopen.76073.

[11] A. Porras, A. Maranon, I. A. Ashcroft, Thermo-mechanical characterization of Manicaria saccifera natural fabric reinforced poly-lactic acid composite lamina, Compos. Part A Appl. Sci. Manuf. 81 (2016) 105-110. doi:10.1016/j.compositesa.2015.11.008.

[12] A. Porras, A. Maranon, I. A. Ashcroft, Optimal tensile properties of a Manicaria-based biocomposite by the Taguchi method, Compos. Struct. 140 (2016) 692-701. doi:10.1016/j.compstruct.2016.01.042.

[13] A. K. F. Oliveira, J. R. M. D'Almeida, Characterization of Ubuçu (Manicaria saccifera) Natural Fiber Mat, Polym. from Renew. Resour. 5 (2014) 13-28. doi:10.1177/204124791400500102.

[14] A. Oliveira, J. D'Almeida, Description of the mechanical behavior of different thermoset composites reinforced with Manicaria saccifera fibers, J. Compos. Mater. 48 (2014) 1189-1196. doi:10.1177/0021998313484622.

[15] E. Kaswell, Wellington Sears Handbook of Industrial Textiles, 1st ed., Wellington Sears Company, Inc., New York, N.Y., 1963.

[16] R. Kandimalla, S. Kalita, B. Choudhury, D. Devi, D. Kalita, K. Kalita, S. Dash, J. Kotoky, Fiber from ramie plant (Boehmeria nivea): A novel suture biomaterial, Mater. Sci. Eng. C. 62 (2016) 816-822. doi:10.1016/j.msec.2016.02.040.

[17] A. K. M. Moshiul Alam, M. D. H. Beg, D. M. Reddy Prasad, M. R. Khan, M. F. Mina, Structures and performances of simultaneous ultrasound and alkali treated oil palm empty fruit bunch fiber reinforced poly(lactic acid) composites, Compos. Part A Appl. Sci. Manuf. 43 (2012) 1921-1929. doi:10.1016/j.compositesa.2012.06.012.

[18] J. A. Ryan, Evolution of Cell Culture Surfaces, BioFiles. 3.8 (2008) 21.

[19] K. L. Tovar-Carrillo, K. Nakasone, S. Sugita, M. Tagaya, T. Kobayashi, Effects of sodium hypochlorite on Agave tequilana Weber bagasse fibers used to elaborate cyto and biocompatible hydrogel films, Mater. Sci. Eng. C. 42 (2014) 808-815. doi:10.1016/j.msec.2014.06.023.

[20] Q. Liu, L. Luo, L. Zheng, Lignins: Biosynthesis and Biological Functions in Plants, Int. J. Mol. Sci. 19 (2018) 335. doi:10.3390/ijms19020335.

[21] A. T. Wood, D. Everett, K. I. Budhwani, B. Dickinson, V. Thomas, Wet-laid soy fiber reinforced hydrogel scaffold: Fabrication, mechano-morphological and cell studies, Mater. Sci. Eng. C. 63 (2016) 308-316. doi:10.1016/j.msec.2016.02.078.

[22] W. G. Brodbeck, G. Voskerician, N. P. Ziats, Y. Nakayama, T. Matsuda, J. M. Anderson, In vivo leukocyte cytokine mRNA responses to biomaterials are dependent on surface chemistry, J. Biomed. Mater. Res. 64A (2003) 320-329. doi:10.1002/jbm.a.10425.

[23] J. D. Humphrey, E. R. Dufresne, M. A. Schwartz, Mechanotransduction and extracellular matrix homeostasis, Nat. Rev. Mol. Cell Biol. 15 (2014) 802-812. doi:10.1038/nrm3896.

[24] S. Bringman, J. Conze, D. Cuccurullo, J. Deprest, K. Junge, B. Klosterhalfen, E. Parra-Davila, B. Ramshaw, V. Schumpelick, Hernia repair: the search for ideal meshes, Hernia. 14 (2010) 81-87. doi:10.1007/s10029-009-0587-x.

[25] C. T. Laurencin, J. W. Freeman, Ligament tissue engineering: An evolutionary materials science approach, Biomaterials. 26 (2005) 7530-7536. doi:10.1016/j.biomaterials.2005.05.073.

[26] N. Yamamoto, K. Ohno, K. Hayashi, H. Kuriyama, K. Yasuda, K. Kaneda, Effects of Stress Shielding on the Mechanical Properties of Rabbit Patellar Tendon, J. Biomech. Eng. 115 (1993) 23. doi:10.1115/1.2895466.

[27] H. Cheung, M. Ho, K. Lau, F. Cardona, D. Hui, Natural fibre-reinforced composites for bioengineering and environmental engineering applications, Compos. Part B Eng. 40 (2009) 655-663. doi:10.1016/j.compositesb.2009.04.014.

[28] F. Namvar, M. Jawaid, P. M. Tahir, R. Mohamad, S. Azizi, A. Khodavandi, H. S. Rahman, M. D. Nayeri, Potential use of plant fibres and their composites for biomedical applications, BioResources. 9 (2014) 5688-5706.

[29] H. Yamada, Strength of Biological Materials, Williams & Wilkins, Baltimore, 1970.

[30] E. Gentleman, A. N. Lay, D. A. Dickerson, E. A. Nauman, G. A. Livesay, K. C. Dee, Mechanical characterization of collagen fibers and scaffolds for tissue engineering, Biomaterials. 24 (2003) 3805-3813. doi:10.1016/S0142-9612(03)00206-0.

[31] A. Goins, A. R. Webb, J. B. Allen, Multi-layer approaches to scaffold-based small diameter vessel engineering: A review, Mater. Sci. Eng. C. 97 (2019) 896-912. doi:10.1016/j.msec.2018.12.067.

[32] D. R. Sumner, Long-term implant fixation and stress-shielding in total hip replacement, J. Biomech. 48 (2015) 797-800. doi:10.1016/j.jbiomech.2014.12.021.

What is claimed is:

1. A method of growing mammalian tissue comprising:
providing a biocompatible textile mesh made from a naturally woven fiber mat from a Manicaria saccifera palm bract, wherein the fiber mat has been treated to remove oils and lignin from the surface of palm fibers in the mat; and
seeding the biocompatible textile mesh with a population of mammalian cells, and
growing the cells on the mesh to form a tissue.

2. The method of claim 1, further comprising autoclaving the treated fiber mat to sterilize the mat prior to seeding with the population of cells.

3. The method of claim 2, wherein autoclaving is at a temperature of about 105 to about 134 for a time of about 15 min or more.

4. The method of claim 1, wherein treatment to remove oils and lignin comprises washing with water and an alkali solution to reduce hydrophobicity of the fibers.

5. The method of claim 1, wherein the biocompatible textile mesh consists essentially of a portion of a naturally woven fiber mat cut from a bract of Manicaria saccifera that has been treated with water and alkali.

6. The method of claim 1, wherein the treated fiber mat of the biocompatible textile mesh has not been functionalized with additional proteins.

7. The method of claim 1, wherein growing the cells comprises culturing the seeded mesh in a compatible culture medium in vitro such that the cells grow on the mesh to form a tissue.

8. The method of claim 1, wherein growing the cells comprises implanting or applying the seeded biocompatible textile mesh in or on a subject and growing the cells to form a tissue in vivo in the subject.

9. A method of growing mammalian tissue in vivo comprising:
providing a biocompatible textile mesh made from a naturally woven fiber mat from a Manicaria saccifera palm bract, wherein the fiber mat has been treated to remove oils and lignin from the surface of palm fibers in the mat; and
implanting the biocompatible textile mesh in a mammalian subject or applying the biocompatible textile mesh to a tissue of a mammalian subject, such that the subject's own cells grow on the mesh in vivo to form a tissue.

10. The method of claim 9, wherein the subject is a human subject.

11. The method of claim 9, wherein the subject is in need of treatment for a hernia and the biocompatible mesh is a hernia mesh.

12. The method of claim 9, wherein the biocompatible textile mesh is further seeded with mammalian cells prior to implantation, and wherein the cells are selected from the group consisting of: the subject's own cells, heterologous stem cells, autologous stem cells, allogenic stem cells, and other mammalian cells.

13. The method of claim 9, wherein the biocompatible textile mesh is a wound patch.

14. An engineered, biocompatible tissue construct comprising:
a biocompatible textile mesh comprising a biocompatible textile mesh made from a naturally woven fiber mat from a Manicaria saccifera palm bract, wherein the fiber mat has been treated to remove oils and lignin from the surface of palm fibers in the mat and autoclaved to sterilize the mat; and
a population of living cells growing on the fibers of the mesh.

15. The engineered, biocompatible tissue construct of claim 14, wherein the cells are mammalian cells.

16. The engineered, biocompatible tissue construct of claim 14, wherein the cells are human cells.

17. The engineered, biocompatible tissue construct of claim 16, wherein the human cells are stem cells.

18. The engineered, biocompatible tissue construct of claim 14, wherein the population of cells comprises more than one cell type.

19. The engineered, biocompatible tissue construct of claim 14, wherein the biocompatible textile mesh consists essentially of a portion of a naturally woven fiber mat cut from a bract of Manicaria saccifera that has been treated with water and alkali.

20. The engineered, biocompatible tissue construct of claim 14, wherein the biocompatible textile mesh does not comprise added extracellular matrix proteins, such as collagen, fibronectin, or added adhesion ligands.
